(19) United States
(12) Patent Application Publication
(10) Pub. No.: US 2005/0250216 A1
(43) Pub. Date: Nov. 10, 2005

(54) Automated high-throughput solubility screening of organic compounds by combustion analysis using CO2 detection

(76) Inventor: Dong Cuan Liang, Vancouver (CA)

Correspondence Address: Dong C. Liang, 1001 East Pender Street, Vancouver, V6A 1W2, CA

(21) Appl. No.: 10/838,331

(22) Filed: May 5, 2004

(51) Int. Cl. G01N 33/00

(52) U.S. Cl. 436/139; 436/160

(57) ABSTRACT

Described is a new high-throughput method to measure a carbon containing compounds solubility. It centers on measuring the carbon dioxide gas released during the combustion of a saturated solution. The amount of carbon present (which is related to the carbon dioxide gas released) will be directly related to the amount of drug dissolved in the solution. This method can be automated to allow for a high throughput. An advantage of this present invention is that if the chemical formula of the compound is known, then no reference sample is required as in the case of other similar methods.

### RELATED APPLICATIONS

This application claims the benefit of prior filed Provisional Application No. 60/648,664.

### BACKGROUND OF THE INVENTION

Drug discovery efforts have been increasing dramatically in the past few years. A popular methodology to discover new drugs is to take large chemical compound libraries (up to millions of compounds) and screen all of them for the ability to elicit some pharmaceutical effect in one or more pharmaceutical assay. The compounds making up these libraries are derived from a number of sources including natural sources (for example plants), combinatorial chemistry, or from chemical drug design chemistry. Assays done on these compounds can consist of assays from the following categories: cell based assays, binding assays, other in vitro or in vivo assays, and several others.

With such large libraries of compounds, the major focus has been to develop assays which are fast and efficient—thus the evolution of the phrase high throughput screening or HTS. In order for a compound to be labeled as a potential drug, it must pass several rounds of tests or assays throughout the screening process. As a compound travels through the drug development pipeline, the assays become more and more specific and more clinically relevant (i.e. the tests more closely mimic the situation as if it were occurring in the human body). With such large compound libraries, it is not uncommon for several compounds to make it to the clinical trial stage (or nearly to this stage). A lot of time, resources, and effort have been put into a compound to get to this stage. Unfortunately, it is not uncommon to find a compound at this stage that shows great promise throughout the screening process but will not necessarily make a good drug. This could be for several reasons including: it is rapidly broken down by the body and does not have time to be active in the body, it is broken down into toxic decomposition products, it is not able to be absorbed by the body, or it has a very poor solubility and is simply expelled.

Such problems have led to the need to profile the chemical and physicochemical properties of chemical compounds before or during the compounds progress through the drug discovery pipeline. This profiling includes measuring the solubility of a compound, the rate at which it can cross cell membranes, the rate at which it is broken down by enzymes in the body, pKa values, and the list goes on. Initially, such characteristics were measured after a compound made it to the final stages and if a compound then showed not to have appropriate properities it would be abandoned or chemical modifications attempted. This meant that a lot of resources were invested into a compound that had little or no potential as a drug. Therefore, it is more logical to profile compounds chemical characteristics before the expensive screening processes

However, with libraries of millions of compounds, profiling this many compounds presents a significant challenge. Thus, high-throughput systems are being developed for high-throughput profiling. The present invention is intended as a solution for a high-throughput measurement of a compounds solubility. Briefly, if a compound is not sufficiently soluble it will not be possible for the compound to enter the necessary system of the body.

Current techniques to measure solubility include the following:
1) Turbidimetric Method
2) Nephelometric Method
3) Liquid Chromatography/Mass Spectrometry
4) Ultraviolet Method
5) Nitrogen Detection Method

The turbidimetric and nephelometric methods measure light scattering as precipitates are formed in a solution at the saturation concentration of a particular salt or compound. The turbidimetric method monitors the light that passes through a solution, whereas the nephelometric method monitors the light that is scattered by the solution. The Liquid Chromatography/Mass Spectrometry method measures the amount of compound present in a known volume of a saturated solution in order to determine solubility. The Ultraviolet Method uses ultraviolet absorption to measure the concentration of a saturated solution and the Nitrogen Detection Method uses a chemiluminescent technique to determine the solubility/concentration of substance with known a chemical formula. The nitrogen detection method requires that there is nitrogen present in the structure of the chemical compound.

Both the ultraviolet and the nitrogen detection methods are fully developed for commercial use in high-throughput solubility profiling. The present invention described here presents a novel method to measure compound solubility by combustion of a saturated solution and by the subsequent measurement of the CO₂ that is produced during the combustion. The amount of CO₂ that is produced during the combustion is proportional to the amount of the compound that is in the saturated solution. Using this technique, the present invention proposes to measure the solubility of compounds by first producing a saturated solution and then combusting a known volume in order to determine the amount that is dissolved in the solution.

### SUMMARY OF THE INVENTION

Solubility is the amount of a substance that can be dissolved in a given amount of solvent. Solubility is determined by measuring the amount of a compound in a saturated solution. Saturated solutions are generally produced by placing an excess of a compound (i.e. an amount greater then the solubility) and allowing the solution to become saturated before removing the excess solid. The amount of the compound contained in this sample is then measured, which allows the determination of the solubility of the compound.

Almost all chemical compounds used as drugs in the market today are carbon containing. Thus, by combusting a sample of the compound and by measuring the amount of CO₂ produced during the combustion process, one can determine the amount of the compound present. There is one piece of information that must be known about the compound at this stage, and that is chemical formula, and more importantly the amount of carbon present. One advantage of this over other systems is the fact that no reference sample needs to be done on each compound before the solubility measurement is made. This will greatly increase the potential throughput of our device over other solubility measurement devices. The present invention is set to solve the problem of the high throughput measurement by combustion of compound samples in a specific buffer.

### BRIEF DESCRIPTION OF DRAWINGS

FIG. 1 is an overview of the measurement protocol.

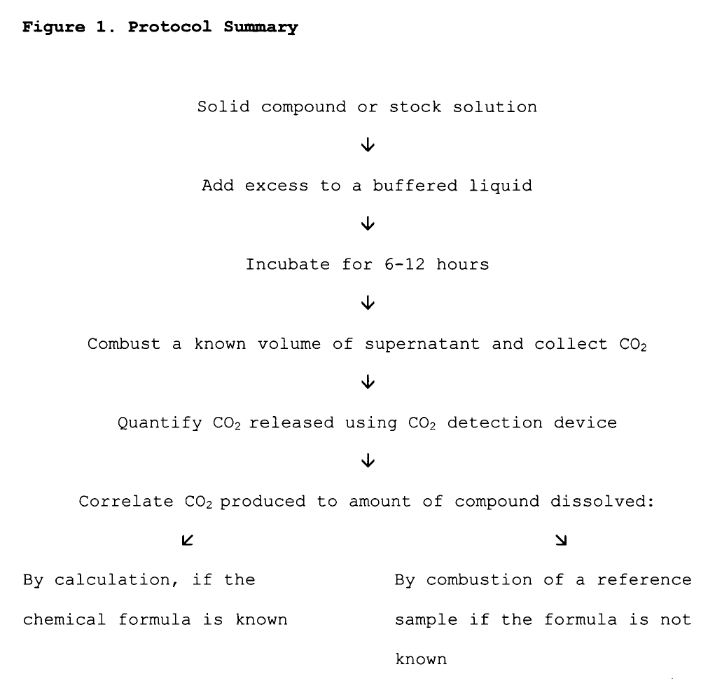

### DETAILED DESCRIPTION OF THE INVENTION

Following is a detailed description of a preferred embodiment of the present invention:

Standard Curve

In order to measure the amount of CO₂ produced by the combustion of a sample, a set of standards with known concentration and carbon content are combusted and the CO₂ produced is measured. This will allow the construction of a standard curve so that when an unknown sample is burned the amount of CO₂ can be correlated to the amount of carbon present and subsequently the amount of sample present. This assumes that you know the concentration of the chemical formula of the compound.

Saturated Sample

In order to measure the solubility of a substance, one needs to produce a saturated sample. This is a sample in which no more of the compound will dissolve. This is produced via the following steps:
1) Add a sufficient amount of the compound (which will be a known mass) of interest to a solution of buffer such that the amount added exceed the solubility.
2) Incubate the sample for a sufficient amount of time to allow saturation to occur.
3) Separate the liquid phase from the excess solid.

It is possible that the entire compound that was added to the buffer solution is dissolved and that a saturated solution was not produced. Thus, the solubility value that is measured may not reflect the true value. In such cases there are two options as to the course of action. First, the experiment could be repeated in the same way except that more of the compound would be added and hopefully a saturated solution produced. Secondly, the solubility of the compound would just be assumed to be greater then the amount added. This assumption is generally sufficient, since drug companies are generally concerned about a minimum solubility—any value higher then this is not an issue.

Combustion

Once a saturated solution has been produced the solid undissolved portion must be separated from the liquid phase. This can be done using a number of techniques including but not limited to filtration, centrifugation, and careful pipeting of the liquid phase. Next, a known amount of the liquid phase needs to be combusted. A known volume is placed into a combustion chamber where the sample is first dehydrated and then combusted. The preferred embodiment of the burning chamber would be an enclosed chamber either made of or containing a metal heating element whose temperature can easily be controlled by varying the electricity supplied to it. There are several metals available which can easily reach 900 to 1100 degrees Celsius, which is sufficient to combust most carbon containing compounds.

Carbon Dioxide Detection

After a sample is combusted, the gases produced would be collected or sent to an instrument or device capable of detecting CO₂ gas. This signal would correspond to an amount of carbon present in the sample, by comparison to the standard curve. This amount of carbon could be used to calculate the amount of the sample that was dissolved in the saturated solution, which would give the solubility of the compound. Note that such a calculation can only be done if the formula of the compound is known. If this is not known, something extra must be done in order to obtain the solubility value. Briefly, a reference sample needs to be produced and combusted to determine the signal produced by a known amount of solid in a known volume. It is important at this stage to add an amount of solid that is much less then the solubility to ensure that all of the compound that is added is dissolved. We can consider this as a one point calibration. However, the measurement will be more accurate and efficient, if the chemical formula is known.

The solution in which all samples are dissolved should be a suitable buffer at or near a physiologically relevant pH. It is important to use the same buffer for any solution that is combusted since they may contain some carbon and would therefore produce some CO₂; it is desirable to have this effect present in any sample that is burned so as to cancel it out.

Protocol Summary

Following is a summary of the experimental in a preferred embodiment which serves to further describe the invention.

A saturated solution of a potential drug is produced by adding an excess of that drug to a suitable pH buffer. The buffer used is one that can maintain the pH at a physiologically relevant pH (this is generally considered to be a pH value of 7.2 to 7.4, but it may be outside this range). This mixture is allowed to incubate for a period of time (approximately 6-12 hrs). The portion of the drug which has not dissolved is removed by filtration, by centrifugation, or by carefully drawing up the supernatant. An aliquot of the saturated solution (1-20 μL) is then placed into a combustion chamber whose temperature is precisely controlled over a specific temperature program. This temperature program will first dry the aliquot and then combust it in a controlled fashion. During the combustion stage, CO₂ gas will be released. Note that it is very important that no extra CO₂ be introduced at this stage (i.e. the air used during the combustion process must be CO₂ free) . The amount of CO₂ released can be quantified by CO₂ gas analysis techniques such as infrared spectroscopy or mass spectrometry. If the chemical formula of the compound was known, the amount of drug that was present in the saturated solution can be quantified, and therefore the solubility can be calculated in units of mass per unit volume. Most of the instruments of today require the production of a standard curve or a one point calibration for each compound in order to measure the drug concentration. If the formula of the compound is known, our instrument does not require this calibration for each compound making it inherently faster. In such a case our instrument would only require an initial calibration by using either gaseous CO₂ or by combusting a solution of an organic compound of known concentration and known chemical formula. We have achieved good results using tartrazine as a universal standard.

If there was no knowledge of the compounds formula, a technique must be employed to produce both a sample (the saturated solution) and a reference solution of the same compound (a solution of known concentration). The reference solution can then be used to calculate the concentration in the sample. There are several techniques to produce the reference and a sample solution and the reader is encouraged consult the literature on the topic.

Our device will consist of robotics, liquid handling systems, and various fluidics systems to prepare the saturated solution and any other samples required to enable measurement of the solubility. As stated above, the analysis of these solutions will be carried out by addition of a small aliquot into a combustion chamber. In one embodiment, the heating element will be a band of suitable metal (such as nickel chrome alloys) connected to a power supply and the required electronics. By controlling the current through the metal band, we can control its temperature. The temperature of the band will be changed to dry and eventually combust a sample. This combustion will occur inside of a sealed chamber which has an inlet for a carbon free air supply and an outlet connected to the detector.

During the combustion, the gases produced will be forced out through the outlet by continual addition of carbon free air. This gas flow will then go to a CO₂ detection system that will continually monitor the CO₂ output. The amount of CO₂ released can then be quantified and is directly related to the concentration of drug in the solution. The throughput of our device can be increased by using a series of combustion and detection setups to do simultaneous measurements.

1. A method to measure the aqueous solubility of a carbon containing chemical compound comprising:
a. producing a saturated solution of the said compound in a buffered liquid such that the maximum amount of said compound is dissolved in said buffered liquid;
b. placing a known volume of said saturated solution into a combustion chamber such that the said compound is combusted to produce carbon dioxide gas;
c. collecting the said carbon dioxide gas and measuring the concentration of carbon dioxide using a carbon dioxide measuring device; and
d. determining the amount of said compound that was present in the said known volume of the said saturated solution by calculation.

2. The method of claim 1, wherein the said combustion chamber contains an electrical heating element.

3. The method of claim 1, wherein the said calculation involves comparing the carbon dioxide gas produced by the said saturated solution during combustion to a standard curve.

4. The method of claim 3, wherein the said standard curve is produced by combustion of known amounts of the said compound when the chemical formula of the said compound is not known or by combustion of a carbon containing chemical with a known chemical formula when the formula of the said compound is known.

5. The method of claim 1, wherein the saturated solution is produced by addition of an amount of the said compound that is in excess of the solubility; where the said compound is added to the said buffered liquid as a solid or as a concentrated stock solution and is left to incubate for 6-12 hours.

6. The method of claim 5, wherein the said stock solution is made using DMSO or some other suitable solvent in which the said compound has a high solubility.

7. The method of claim 1, wherein the said known volume of the said saturated solution is 1 to 20 micro-liters.

8. The method of claim 1, wherein during the formation of the saturated solution a portion of the said compound remains as or forms a solid which is separated from the said saturated solution.

9. The method of claim 1, wherein the said buffered liquid maintains the pH at a physiologically relevant pH.

10. The method of claim 1, wherein the said combustion chamber is capable of heating the sample to at least 900 degrees Celsius.

11. The method of claim 1, wherein the said carbon dioxide measuring device is a mass spectrometer.

12. The method of claim 8, wherein the said mass spectrometer is selected from the group fourier-transform mass spectrometer, ion-trap mass spectrometer, magnetic-sector mass spectrometer, quadrupole mass spectrometer, and time of flight mass spectrometer.

13. The method of claim 1, wherein the said carbon dioxide measuring device is an infrared spectrometer.

14. The method of claim 1, wherein the said buffered liquid is phosphate buffered saline.

15. The method of claim 1, wherein all the steps are done using robotics, machinery, and automated liquid handling devices.

16. The method of claim 1, wherein the combustion of the said compound occurs using a gas supplied that does not contain any carbon dioxide.

17. The method of claim 12, wherein the said robotics, machinery, and liquid handling devices are computer controlled.

18. The method of claim 8, wherein the said solid formed is separated from the supernatant using one of centrifugation or filtration.
